(19) United States
(12) Patent Application Publication
(10) Pub. No.: US 2012/0259357 A1
(43) Pub. Date: Oct. 11, 2012

(54) Epistaxis Apparatus

(76) Inventor: Peter C. Elliott, Manitou Springs, CO (US)

(21) Appl. No.: 13/082,152

(22) Filed: Apr. 7, 2011

(51) Int. Cl. A61M 29/00

(52) U.S. Cl. 606/196

(57) ABSTRACT

Apparatus and methods for treating an epistaxis condition are disclosed herein. An exemplary epistaxis apparatus typically comprises an elongated body, a first conduit, and a second conduit. The elongated body includes a proximal end, a longitudinal axis, and a distal end. In use, the proximal end of the epistaxis apparatus is introduced into a patient's nostril. The distal end can be operative coupled to a suction source or a light source. Each of the first and second conduits is generally tubular having a proximal opening and distal opening. An exemplary light conducting apparatus comprise a generally frustoconical section, a generally elongated cylindrical section, and an angled tip section having a light conducting tip. The light conducting apparatus is typically adapted to operatively couple with an otoscope. Exemplary methods utilizing various embodiments of both the epistaxis apparatus and the light conduction apparatus are provided.

### FIELD OF THE INVENTION

The present invention relates generally to apparatus and methods to treat an epistaxis condition. More particularly, the present invention relates to apparatus and methods to treat an epistaxis condition using cauterization.

### BACKGROUND

Epistaxis (i.e., a nosebleed or bleeding from the nostril) is a common medical condition. There are typically two types of nosebleeds: an anterior nosebleed and a posterior nosebleed. Anterior nosebleeds are the most common of the two and are often occur as a result of trauma to the nose or dry nasal membranes. Dry climates and high altitudes, as well as the administration of certain medications like aspirin, nose drops, and blood thinners, can also contribute to the frequency and duration of nosebleeds. Moreover, various ailments such as, but not limited to, hemophilia, high blood pressure, arteriosclerosis, and liver disease can also cause or exacerbate an epistaxis condition.

Proper treatment of an epistaxis condition is difficult to perform because of the amount of bleeding that occurs and the location of the bleeding vessel(s). The treatment is made more challenging due to the fact that usually a single person (typically a physician or other health care provider) is required to perform the proper corrective procedure. Prior art methods used to treat epistaxis conditions include applying pressure and packing, as well as, performing cautery. However, to successfully treat an epistaxis condition, excess blood must be removed from the general area and the source of the bleeding must be clearly visible to the physician while the cauterization is being performed.

Given the plurality of simultaneous actions required to perform a proper cauterization and treatment of an epistaxis condition, it would be advantageous to design apparatus and devise methods that enable a physician to simultaneously perform the necessary actions using only a single hand or, at most, both hands during the entire procedure.

### BRIEF DESCRIPTION OF THE FIGURES

FIG. 1 is a perspective view of an epistaxis apparatus according to an embodiment.

FIG. 2 is a side perspective view of an epistaxis apparatus according to an embodiment.

FIG. 3 is a top perspective view of an epistaxis apparatus according to an embodiment.

FIG. 4 is a perspective view of a light conducting apparatus for use during treating an epistaxis condition according to an embodiment.

FIG. 5 is a perspective view of a light conducting apparatus coupled to a speculum for use during treating an epistaxis condition according to an embodiment.

### DETAILED DESCRIPTION

Embodiments of the present invention comprise epistaxis apparatus, light conducting apparatus, and methods for treating an epistaxis condition. An exemplary embodiment of an epistaxis apparatus typically comprises an elongated body, a first conduit, and a second conduit. The elongated body includes a proximal end, a longitudinal axis, and a distal end. In use, the proximal end of the epistaxis apparatus is introduced into the patient's nostril. The distal end can be operatively coupled to a suction source or a light source depending on the particular embodiment.

Each of the first and second conduits is generally tubular having a proximal opening and distal opening. Each conduit runs the length, and in the general longitudinal direction, of the elongated body. The first conduit includes a longitudinal opening that allows the interior of the conduit to be accessed. Thus, when a cautery source is disposed substantially within the first conduit, a shaft portion of the cautery source can be accessed thereby enabling a user (typically a physician or other health care provider) to laterally move the cautery source as necessary during the procedure to treat the epistaxis condition. The second conduit can include an aperture to control the suction force when a suction source is operatively coupled to the distal opening of the second conduit. The user can cover or uncover the aperture to effectively turn the suction force on or off at the proximal opening of the second conduit.

In some embodiments, all or part of the epistaxis apparatus are disposable. The cautery source can be a silver nitrate stick fitted into and secured by the first conduit. The first conduit provides stability and support to a cautery stick thereby lessening the risk of it snapping, while also allowing the cautery source to be easily removed and replaced. In use, embodiments of the epistaxis apparatus are held in a similar manner as a pen or pen-like device whereby the user can utilize his or her fingers to operate the apparatus (e.g., use a finger to slide the cautery source back and forth and a finger tip to cover or uncover the aperture in the second conduit). The elongated body of the epistaxis apparatus can have a seat or flanged area framing the longitudinal opening and cutout section whereby the user can more easily secure/control the cautery source as well as rest his or her finger during procedure.

Embodiments of a light conducting apparatus comprise a generally frustoconical section, a generally elongated cylindrical section, and an angled tip section having a light conducting tip. The light conducting apparatus is typically adapted to operatively couple with an otoscope. The generally frustoconical section of the light conducting apparatus is adapted to be fitted and secured in an otoscope speculum.

Features and advantages of the embodiments of the epistaxis apparatus and light conducting apparatus make the procedure to properly treat an epistaxis condition easy, efficient, and effective. An exemplary epistaxis apparatus fits into the user's hand and has at least two functions (suction, light, and/or cautery source) combined into a one thin easily-grasped device. Methods using various embodiments of the epistaxis apparatus and light conducting apparatus are disclosed. In some methods, both the epistaxis apparatus and light conducting apparatus are used to treat an epistaxis condition. Use of the apparatus and methods disclosed herein greatly increase the efficiency and effectiveness of the epistaxis procedure and the probability of a successful outcome.

### Terminology

The terms and phrases as indicated in quotes (“ ”) in this section are intended to have the meaning ascribed to them in this Terminology section applied to them throughout this document including the claims unless clearly indicated otherwise in context. Further, as applicable, the stated definitions are to apply, regardless of the word or phrase's case, to the singular and plural variations of the defined word or phrase.

The term “or” as used in this specification and the appended claims is not meant to be exclusive rather the term is inclusive meaning: either or both.

References in the specification to “one embodiment”, “an embodiment”, “an alternative embodiment” and similar phrases mean that a particular feature, structure, or characteristic described in connection with the embodiment is included in at least an embodiment of the invention. The appearances of the phrase “in one embodiment” in various places in the specification are not necessarily all meant to refer to the same embodiment.

The term “couple” or “coupled” as used in this specification and the appended claims refers to either an indirect or direct connection between the identified elements, components or objects. Often the manner of the coupling will be related specifically to the manner in which the two coupled elements interact.

The term “removable,” “removably coupled,” “readily removable,” “threadably coupled,” and similar terms, as used in this specification and appended claims, refer to structures that can be uncoupled from an adjoining structure with relative ease (i.e., non-destructively and without complicated or time consuming process), and can also be readily reattached or coupled to the previously adjoining structure.

Directional and/or relationary terms such as, but not limited to, left, right, top, bottom, vertical, horizontal, back, front and lateral are relative to each other and are dependent on the specific orientation of an applicable element or article, and are used accordingly to aid in the description of the various embodiments and are not necessarily intended to be construed as limiting.

As applicable, the terms “about” and “generally” as used herein unless otherwise indicated mean a margin of +−20%. Also, as applicable, the term “substantially” as used herein unless otherwise indicated means a margin of +−10%. Concerning angular measurements, “about” or “generally” refer to +−10 degrees and “substantially” refers to +−5.0 degrees unless otherwise indicated. It is to be appreciated that not all uses of the above terms are quantifiable such that the referenced ranges can be applied.

### An Exemplary Embodiment of an Epistaxis Apparatus

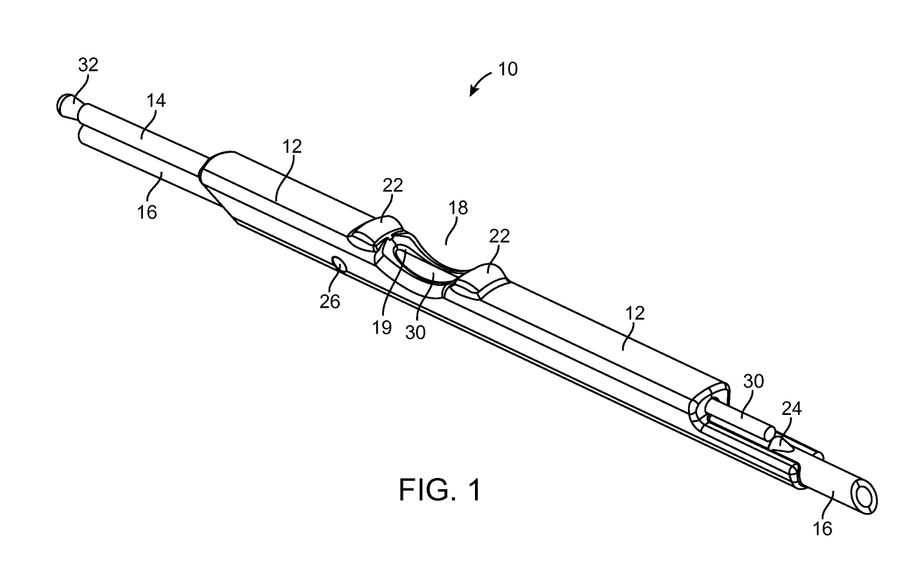

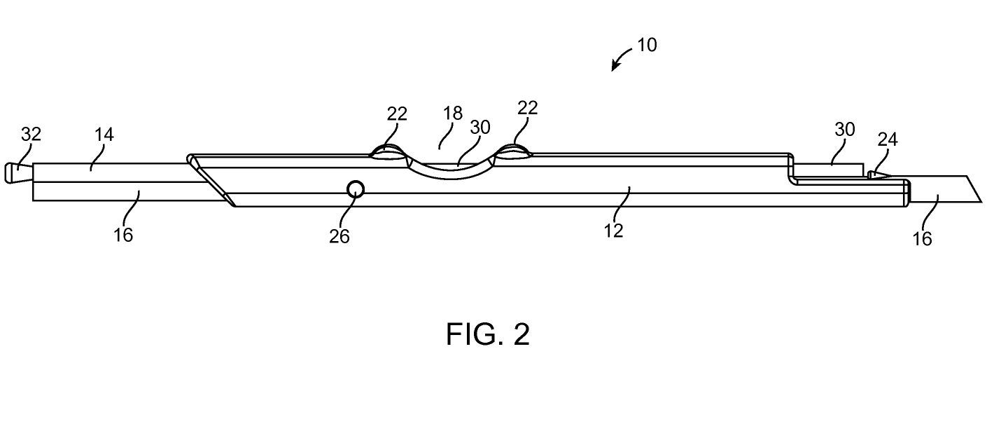

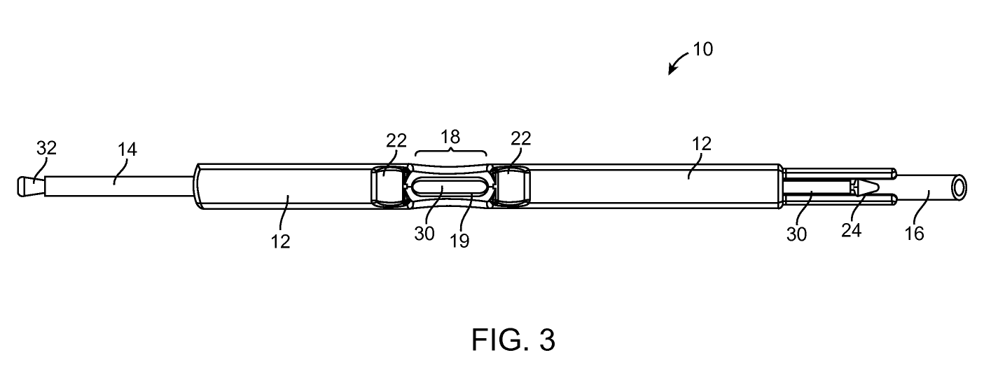

An exemplary embodiment of an epistaxis apparatus 10 is illustrated in FIGS. 1 through 3. The epistaxis apparatus 10 typically comprises an elongated body 12, a first conduit 14, and a second conduit 16. The elongated body 12 includes a proximal end, a longitudinal axis, and a distal end. In use, the proximal end of the epistaxis apparatus 10 is introduced into the patient's nostril whereas the distal end can be operative coupled to a suction source or a light source, for instance. As can be seen in FIG. 1, the proximal end of the epistaxis apparatus 10 is where the head portion 32 of the chemical cautery stick 30 is generally located and the distal end is where the stopper 24 and extended end of the second conduit 16 are generally located.

The first conduit 14 has a proximal opening and a distal opening substantially aligned with the proximal end and distal end of the elongated body 12. The first conduit 14 also includes a longitudinal opening 19. As illustrated, the longitudinal opening 19 is typically disposed around the center of the first conduit 14 nearer the proximal end thereof. However, the longitudinal opening 19 can be disposed in various locations along the longitudinal axis of the first conduit 14. The longitudinal opening 19 exposes at least a portion of the interior area of the first conduit 14. In operation, the longitudinal opening 19 allows the user of the epistaxis apparatus 10 to contact the cautery source device, typically a chemical cautery stick 30, such that the cautery source device can be moved forward or backward during the cauterization process.

At least a portion of the first conduit 14 is coupled to the elongated body 12. The first conduit 14 is typically permanently affixed to the elongated body 12. However, in some embodiments, the first conduit 14 may be removably coupled to the elongated body 12. Additionally, while the first conduit 14 can be constructed from various materials, at least one version of the first conduit is comprised of metal. It is also pertinent to note that the longitudinal axis of the first conduit 14 extends generally parallel to the longitudinal axis of the elongated body 12.

In some embodiments and as illustrated in the Figures, the elongated body 12 also includes a cutout section 18. The cutout section 18 is typically disposed along a top edge of the elongated body 12 and exposes (or, in some embodiments, extends into) the longitudinal opening 19 of the first conduit 14. Hence, the longitudinal opening 19 is substantially aligned with the cutout section of the elongated body 12. One or more bulbous flanges 22 may surround the cutout section 18 so that the user may comfortably rest his or her finger within the cutout section 18. In some embodiments, the cutout section 18 can be operatively coupled to a rotatable disk or the like, with which the user can roll the back and forth to provide a means of longitudinally moving the cautery source within the first conduit 14.

The second conduit 16 similarly has a proximal opening and a distal opening substantially aligned with the proximal end and distal end of the elongated body 12. Additionally, the second conduit 16 typically includes an aperture 26. The aperture 26 extends through at least the second conduit 16; however, as illustrated, the aperture 26 can extend through a side wall of the elongated body 12 prior to extending through the second conduit 16. The aperture 26 exposes at least a portion of the interior area of the second conduit 16.

In operation, the distal opening of the second conduit 16 is typically operatively coupled to a suction source. The distal opening can include a connector end adapted to more readily couple with a standard suction tube of the suction source as would be found in a doctor's office or hospital, for instance. Hence, the proximal end of the second conduit 16 provides a suction end of the epistaxis apparatus 10. The aperture 26 allows the user of the epistaxis apparatus 10 to control the suction force by either covering or uncovering the aperture 26 with his or her finger. For example, when the aperture 26 is covered and the suction source is activated, the proximal opening of the second conduit 16 will provide suction force to remove blood and mucus from the patient's nostril. This helps enable the user to better see which blood vessel or vessels on the inner surface of the nostril have ruptured and are in need of cauterization. Once the blood, blood clots, mucus, and the like has been cleared from the general area within the nostril, the aperture 26 can be uncovered thereby eliminating or substantially reducing the suction force applied to the proximal end of the second conduit 16. With the suction force eliminated or substantially reduced, the tip of the cautery source can be more accurately placed against the ruptured blood vessel or vessels.

As described above, embodiments of the epistaxis apparatus 10 typically include and/or are adapted to receive a cautery source. The cautery source is typically inserted into the first conduit 14. The cautery source can be a chemical cautery stick 30 having a head portion 32 and a shaft portion. However, in some embodiments, the cautery source can be an electrical cautery probe. The chemical cautery stick 30 can be a silver nitrate cautery stick; however, other types of chemical cautery sticks having various chemicals and caustic agents are contemplated in other embodiments.

Moreover, embodiments of the epistaxis apparatus 10 (particularly those which are adapted to receive a chemical cautery stick 30) typically include a stopper 24. The stopper can be positioned on the second conduit 16 near the distal end of the first conduit or another suitable location on the elongated body 12. The stopper 24 is positioned as to prevent the distal end of the shaft portion of the chemical cautery stick 30 from sliding or being moved too far toward the distal end of the epistaxis apparatus 10. Thus, the head portion 32 and chemicals/caustic agents thereon will avoid being damaged by the proximal end of the first conduit 14 as the head portion 32 may be of a slightly larger diameter that the inner diameter of the first conduit 14. In other variations the epistaxis apparatus 10, the stopper may simply be a closure of the distal opening of the first conduit 14.

The elongated body 12 is typically, but not necessarily, comprised of polypropylene plastic. As illustrated, the elongated body 12 can be a combination of two or more pieces that generally surrounds a large portion of both the first and second conduits 14, 16 in order to secure the conduits. However, in some embodiments the elongated body can be an integrated unit with one or more of the conduits. Moreover, other embodiments are contemplated whereby only a small portion of the eternal surface area of the first and second conduit is in contact with the elongated body. In such embodiments, the elongated body would basically serve to minimally couple or connect the first and second conduits together and may even be a significantly smaller clip-like mechanism. In other embodiments of the epistaxis apparatus, the first conduit may be formed in whole or part by a longitudinal aperture through the elongated body. In such an embodiment, the longitudinal opening of the first conduit and the cutout section of the elongated body may be one and the same. For example, the longitudinal opening of the first conduit may be formed from the cutout section of the elongated body when the elongated body actually defines a portion (typically a middle portion) of the first conduit.

Although various lengths and sizes are contemplated, in at least one embodiment, the epistaxis apparatus 10 measures approximately 6 inches in total length (i.e., the longest of the longitudinal distances from the proximal to distal ends of the first and second conduits 14, 16 and/or elongated body 12). In an embodiment, the longest longitudinal distance of the epistaxis apparatus 10 extends from the proximal ends of both the first and second conduits 14, 16 to the distal end of the second conduit 16. Additionally, the general diameter of the epistaxis apparatus 10 measures approximately 0.375 inches (i.e., the general diameter or cross-sectional area of the elongated body 12) in at least one embodiment. The diameter of each of the first and second conduits can be approximately 0.125 inches in such an embodiment.

In at least one embodiment, the epistaxis apparatus 10 is a disposable, pre-sterilized unit. However, in other embodiments, some portions of the epistaxis apparatus 10 may be disposable (e.g., one of the conduits 14, 16 when removably coupled from the elongated body 12) and some portions may be constructed to be and used in a permanent or semi-permanent fashion. Additionally, all or part of the epistaxis apparatus may be created from an injection mold or other efficient construction means.

It is also pertinent to note that some embodiments of the epistaxis apparatus include a third conduit. The third conduit typically includes a proximal opening and a distal opening. The third conduit can be substantially similar in length as first and/or second conduit. The diameter of the third conduit may vary depending on what implement is to be placed therein. At least a portion of the third conduit is attached to the elongated body and the three conduits may be arranged in a planar or triangular manner. For example, a three conduit epistaxis apparatus can include a cautery source substantially disposed in the first conduit, a suction source removably coupled to the distal opening of the second conduit, and a sufficiently small and thin light conducting element substantially disposed within the third conduit. The light conducting element has similar characteristic to the light conducting apparatus disclosed below with the exception that it is adapted to fit substantially with a conduit of the epistaxis apparatus and is adapted to receive light from a source other than directly from an otoscope speculum. Hence, the user of such an embodiment can clear the nostril area from blood and mucus and thoroughly inspect the area prior to cauterizing the ruptured blood vessel or vessels.

### An Exemplary Embodiment of a Light Conducting Apparatus

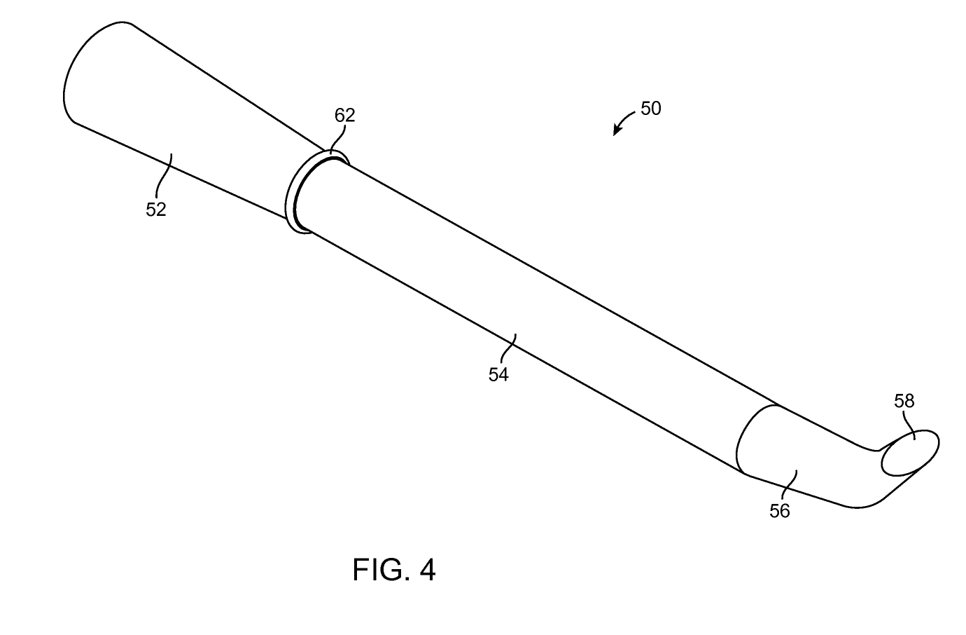

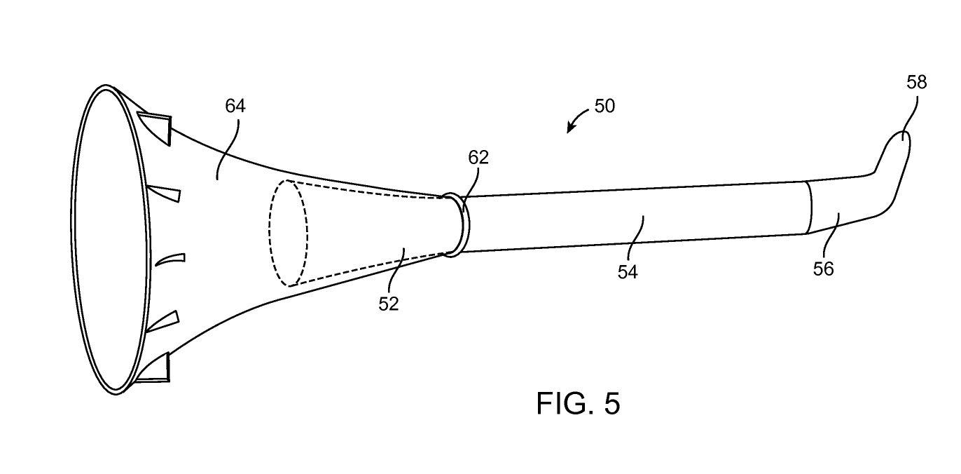

An exemplary embodiment of a light conducting apparatus 50 is illustrated in FIGS. 4 and 5. The light conducting apparatus 50 is typically used during an epistaxis condition in conjunction with an otoscope. The light conducting apparatus 50 comprises a generally frustoconical section 52, a generally elongated cylindrical section 54, and an angled tip section 56 having a light conducting tip 58. In use, generally frustoconical section 52 of the light conducting apparatus 50 is fitted into an otoscope speculum 64. Then, the angled tip section 56 and at least a portion of the generally elongated cylindrical section 54 are introduced into a patient's nostril. The light conducting apparatus 50 is typically comprised of a light conducting plastic; however, other materials such as, but not limited to, glass are contemplated for various embodiments. Moreover, in some embodiments, the light conducting apparatus 50 may be comprised of a color-infused light conducting material.

The generally frustoconical section 52 is suitably sized and adapted to fit within the otoscope speculum 64 for which it is designed. As can be appreciated by one having ordinary skill in the art, the generally frustoconical section 52 can be adapted to fit a variety of otoscope specula. Additionally, the light conducting apparatus 50 typically includes a circumferential flange 62. The circumferential flange 52 is disposed between the generally frustoconical section 52 and the generally elongated cylindrical section 54, and provides stability for the light conducting apparatus 50 when it is inserted into the otoscope speculum 64. The otoscope speculum 64 typically has a wider distal opening that connects to the otoscope and a smaller proximal opening that is inserted into or proximal an orifice of the patient. The light conducting apparatus 50 can be operatively coupled to the otoscope speculum 64 by first passing the angled tip section 56 though the larger distal opening and then through the smaller proximal opening of the otoscope speculum 64. The angled tip section 56 and the generally elongated cylindrical section 54 pass though the smaller proximal opening. The light conducting apparatus 50 becomes sufficiently securely lodged into the otoscope speculum 64 when the circumferential flange 62 passes though the smaller proximal opening and at laeast a portion of the outer surface of the generally frustoconical section 52 is in contact with the inner wall of the otoscope speculum 64.

The circumferential flange 62 is typically comprised of the same light conducting plastic, which has some semi-flexible characteristics allowing it to be slightly compressed as it passes through the proximal opening of the otoscope speculum 64. However, other embodiments are contemplated whereby the circumferential flange is comprised of a semi-flexible material other than the the light conducting material that is used for the majority of the light conducting apparatus 50.

In at least one embodiment, the light conducting apparatus 50, having a longitudinal axis running the lengthwise through the center of the generally elongated cylindrical section 54, is overall approximately 2.55 inches in length. The generally frustoconical section 52 can be 0.65 inches in length with its general diameter gradually decreasing from 0.25 inches at its distal end to 0.17 inches at its proximal end (abutting circumferential flange 62 and the generally elongated cylindrical section 54). The generally elongated cylindrical section 54 can be 1.50 inches in length while having a uniform diameter of 0.17 inches. The circumferential flange 62 can have a diameter of 0.20 inches thereby extending slightly beyond the diameter of the generally elongated cylindrical section 54 and the generally frustoconical section 52 at its proximal end. The angled tip section 56 can be 0.50 inches in length and can have slightly taper for the first 0.30 inches and angle for the next 0.20 inches as the section progresses toward the light conducting tip 58. The angle in at least a portion of the angled tip section 56 can be a 45 degree angle from the longitudinal axis thereby allowing the user to clearly see the inner surface/wall of the patient's nostril. Additionally, the angled tip section 56 is often used to open a patient's nostril by effectively hooking a rim portion of the nostril and gently pulling away from the patient's nose. Hence, the light conducting tip 58 will still be substantially pointed inside the nostril to illuminate the inner surface of the nasal cavity while the patient's nostril is kept open for insertion of another device, such as an epistaxis apparatus, for instance. Moreover, the light conducting tip 58 may be rounded in a slightly convex manner to spread the emitted light in a slightly greater area as well as reduce any discomfort should the tip contact the inner surface/wall of the patient's nostril. Additionally, the specific dimensions listed in this paragraph are exemplary of one embodiment and not intended to limit the scope of the disclosure herein.

It is also pertinent to note that, in order to properly guide the light from the otoscope, the majority of the surface of the light conducting apparatus 50 can contain a different index of refraction than the end portions. For example, an exterior surface or cladding material of the generally elongated cylindrical section 54, circumferential flange 62, and all but the light conducting tip 58 of the angled tip section 56 may have a lower index of refraction that a transparent core material of the light conducting apparatus 50. Hence, light may be kept in the transparent core material causing the light conducting apparatus 50 acts as a waveguide to transmit light between its two end portions. One end portion can be the end of the generally frustoconical section 52 or the generally frustoconical section 52 itself since it will typically resides within otoscope speculum 64 during use. The other end portion is typically the light conducting tip 58, which is angled in some fashion to direct the light to an area of interest within the nostril or other orifice.

### Exemplary Methods of Treating an Epistaxis Condition

New and useful exemplary methods of treating an epistaxis condition using an epistaxis apparatus and/or a light conducting apparatus are disclosed herein. It is to be understood that various embodiments of both the epistaxis apparatus and the light conduction apparatus can be used in conjunction with the exemplary methods. For exemplary purposes, a chemical cautery epistaxis apparatus will be used in the methods disclosed. Additionally, a light conducting apparatus operatively coupled to an otoscope will also be used in some of the methods disclosed. It is to be understood that the epistaxis apparatus 10 as illustrated in FIGS. 1 through 3 and described herein can be used with the exemplary methods of treating an epistaxis condition. Moreover, the light conducting apparatus 50 as illustrated in FIGS. 4 and 5 and described herein can also be used. However, various alternative embodiments and variation of the apparatus and elements thereof can also be in conjunction with the exemplary methods.

The chemical cautery epistaxis apparatus is generally configured to be introduced into a nostril. One embodiment of the chemical cautery epistaxis apparatus typically comprises an elongated body, a first conduit, and a second conduit. The elongated body includes a proximal end, a longitudinal axis, and a distal end. Additionally, a cutout section is disposed along a length of the elongated body. The first conduit has a proximal opening and a distal opening substantially aligned with the proximal end and distal end of the elongated body. The first conduit also includes a longitudinal opening typically disposed around the center of the first conduit nearer the proximal end thereof. A chemical cautery stick is substantially disposed within the first conduit whereby a head portion remains outside of the proximal end of the first conduit and the shaft portion remains substantially within the first conduit. The longitudinal opening is generally aligned with the cutout portion of the elongated body exposes at least a portion of the interior area of the first conduit and the shaft portion of the chemical cautery stick therein.

The second conduit of the chemical cautery epistaxis apparatus similarly has a proximal opening and a distal opening substantially aligned with the proximal end and distal end of the elongated body. In some embodiments of the chemical cautery epistaxis apparatus, the second conduit includes an aperture. The aperture extends through at least the second conduit, but can also extend through a side wall of the elongated body prior to extending through the second conduit. The aperture exposes at least a portion of the interior area of the second conduit and can be used to control the suction force at the proximal end of the second conduit when a suction source is applied to the distal end of the second conduit. However, in some embodiments, the aperture may not be included in the second conduit, particularly when the chemical cautery epistaxis apparatus is designed to receive a light conducting element.

One exemplary method of treating an epistaxis condition, the chemical cautery epistaxis apparatus includes the chemical cautery stick in the first conduit and the light conducting element in the second conduit. A user holds the chemical cautery epistaxis apparatus in a first hand. The chemical cautery epistaxis apparatus is typically held by the user in a similar manner like a pen or pen-like device. Next, a light source is operatively coupled to the light conduction element. The light source can be a small, battery powered LED device that can be attached to the distal end of the second conduit, for instance. Next, the proximal end of the chemical cautery epistaxis apparatus is inserted into the patient's nostril. At least a portion of the proximal end of the chemical cautery epistaxis apparatus enters the nostril when the cautery epistaxis apparatus is equipped with a light conducting element in the second conduit. However, depending on the location of the ruptured blood vessel(s), the proximal end of the second conduit may remain outside of the nostril opening while the head portion of the chemical cautery stick enters the nostril. Light can then be applied via the light conducting element into the nostril so that the user can clearly see the cause of the epistaxis condition and cauterize the appropriate area. The application of the chemicals/caustic agents on the head portion of the chemical cautery stick can be controlled by a finger of the user's first hand. By sliding the shaft portion of the chemical cautery stick accessed through the cutout section and longitudinal opening in the first conduit, the lateral movement of the chemical cautery stick can be controlled. Then, the head portion of the chemical cautery stick can be carefully moved so as to touch the inner surface of the nasal cavity at the location of the ruptured blood vessel(s).

In another exemplary method of treating an epistaxis condition, the chemical cautery epistaxis apparatus includes the chemical cautery stick in the first conduit and an aperture through at least the second conduit. The chemical cautery epistaxis apparatus in such an embodiment can be used to provide suction to remove any blood or mucus while performing the cauterization procedure. Similarly, the user holds the chemical cautery epistaxis apparatus in a first hand. Next, a suction source is operatively coupled to the distal opening of the second conduit. Various suction sources are contemplated for use with the exemplary method. For example, the suction source may include a standard suction tube that can be coupled to the distal opening of the second conduit. Next, the proximal end of the chemical cautery epistaxis apparatus is inserted into the patient's nostril. Both the proximal ends of the first and second conduits typically enter the nostril opening when the cautery epistaxis apparatus is adapted to provide suction via the second conduit. With the suction force activated, suction can be applied to the nostril area via the proximal end of the second conduit thereby removing any blood and/or mucus from the area. The suction force of the proximal end of the second conduit can be can be controlled by the user with a first finger of the user's first hand by covering or uncovering the aperture with the tip of the first finger. Next, the application of the chemicals/caustic agents on the head portion of the chemical cautery stick can be controlled by a second finger of the user's first hand by sliding the shaft portion of the chemical cautery stick accessed through the cutout section and longitudinal opening in the first conduit. Then, the head portion of the chemical cautery stick can be carefully moved so as to touch and cauterize the inner surface of the nasal cavity at the location of the ruptured blood vessel(s). Alternatively a single finger can be used by alternating the finger between the longitudinal opening of the first conduit and the aperture of the second conduit. This exemplary method allows the user to accurately and precisely control the cauterization procedure with a single hand. Thus, the user has his or her second hand available to perform other functions during the cauterization functions.

In yet another exemplary method, an epistaxis apparatus and an otoscope having a light conducting apparatus coupled its speculum are used to treat an epistaxis condition. The epistaxis apparatus can be a chemical cautery epistaxis apparatus comprising a chemical cautery stick in the first conduit and have a suction source operatively coupled to the second conduit. However, other embodiments of the epistaxis apparatus may be used in this exemplary method. The light conducting apparatus can be secured by the speculum of the otoscope and comprise a generally frustoconical section, a generally elongated cylindrical section having a circumferential flange disposed proximal the generally frustoconical section, and an angled tip section having a light conducting tip. However, other embodiments of the light conducting may be used.

With the epistaxis apparatus held in a first hand of the user, the proximal end is inserted into the nostril. Suction can than be then applied by activating the suction source to remove any blood and/or mucus from the area. If the epistaxis apparatus includes an aperture through at least the second conduit, the suction force can be controlled or varied. Nevertheless, the suction force can be controlled by activating or deactivating the suction source. Next, the proximal end of the epistaxis apparatus can be temporarily removed from the nostril opening or moved to a side within of the nostril cavity. With the otoscope held in a second hand, the angled tip section can be inserted into the nostril opening and light applied to the interior of the nostril. Additionally, the angled tip section can be used to hook the nostril end and gently open the nostril wider. Hence, the user can quickly determine the cause of the epistaxis condition and locate any ruptured blood vessel(s) without having to put down the epistaxis apparatus or moving it very much. Lateral movement of cautery source device, such as the chemical cautery stick, can be controlled by accessing and sliding the shaft portion through the longitudinal opening in the first conduit. The cautery head of the cautery source device can then quickly be touched against the inner surface of the naval cavity to cauterize the ruptured blood vessel(s).

An alternative method can be performed whereby the epistaxis apparatus comprised a cautery source in the first conduit and have a light conducting element substantially disposed within the second conduit. Thus, a user can hold the epistaxis apparatus in a first hand to illuminate and cauterize, and hold a standard suction device in a second hand. Thus, the procedure can be preformed efficiently and effectively by the user. It is to be appreciated that many other alternative methods are contemplated as would be obvious to one having ordinary skill in the art given the benefit of this disclosure.

### Alternative Embodiments and Variations

The various embodiments and variations thereof, illustrated in the accompanying figures and/or described above, are merely exemplary and are not meant to limit the scope of the invention. It is to be appreciated that numerous other variations of the invention have been contemplated, as would be obvious to one of ordinary skill in the art, given the benefit of this disclosure.

For example, an embodiment of inventive concept disclosed herein includes: a combination comprising a light conducting apparatus, an otoscope speculum, and an otoscope. Additionally, an embodiment of the epistaxis apparatus is contemplated whereby a nasal speculum is coupled to the proximal end to hold the nostril open during the procedure. In yet another alternative embodiment, the general cutout section area of the elongated body may contain a switch and/or other control mechanisms when the cautery source is an electrical cautery probe.

Moreover, while embodiments of the epistaxis apparatus are typically used in a health care setting like a doctor's office or emergency where a suction source and/or light source is more readily available, self-contained and self-sufficient embodiments of the epistaxis apparatus are contemplated for off premise use (e.g., in an office medical supply kit or first aid kit for sporting events). For example, a suction bulb operatively coupled to and removably attached to the proximal end of the second conduit can provide a suitable suction source for a self-contained epistaxis apparatus.

All variations of the invention disclosed in this application are intended and contemplated to be within the spirit and scope of the invention.

I claim:

1. An epistaxis apparatus configured to be introduced into a nostril, the epistaxis apparatus comprising:
an elongated body having,
a proximal end,
a longitudinal axis,
a cutout section disposed along the length thereof, and
a distal end;
a first conduit having at least a portion coupled to the elongated body and extending generally parallel to the longitudinal axis thereof, the first conduit having a proximal opening, a longitudinal opening substantially aligned with the cutout section of the elongated body, and a distal opening; and
a second conduit having at least a portion coupled to the elongated body and extending generally parallel to the longitudinal axis thereof, the second conduit having a proximal opening and a distal opening.

2. The epistaxis apparatus of claim 1 further comprising a cautery source device substantially disposed within the first conduit.

3. The epistaxis apparatus of claim 2, wherein the cautery source device is a chemical cautery stick.

4. The epistaxis apparatus of claim 3, wherein the chemical cautery stick is a silver nitrate cautery stick.

5. The epistaxis apparatus of claim 1, wherein a one of the elongated body and second conduit further comprises a stopping flange disposed proximal the distal opening of the first conduit.

6. The epistaxis apparatus of claim 1, wherein the cutout section of the elongated body comprises at least one bulbous flange disposed proximal at least a portion of a circumferential edge of the cutout section.

7. The epistaxis apparatus of claim 1 further comprising an aperture through at least the second conduit.

8. The epistaxis apparatus of claim 1 further comprising,
a third conduit having at least a portion coupled to the elongated body and extending generally parallel to the longitudinal axis thereof, the second conduit having a proximal opening and a distal opening.

9. The epistaxis apparatus of claim 8 further comprising,
a cautery source device substantially disposed within the first conduit;
a suction source removable coupled to the distal opening of the second conduit; and
a light conducting element substantially disposed within the third conduit.

10. The epistaxis apparatus of claim 1, wherein the elongated body is substantially comprised of polypropylene plastic.

11. The epistaxis apparatus of claim 1, wherein the first conduit is comprised of metal.

12. A chemical cautery epistaxis apparatus configured to be introduced into a nostril, the chemical cautery epistaxis apparatus comprising:
an elongated body having,
a proximal end,
a longitudinal axis,
a cutout section disposed along a length thereof, and
a distal end;
a first conduit having at least a portion coupled to the elongated body and extending generally parallel to the longitudinal axis thereof, the first conduit having a proximal opening, a longitudinal opening substantially aligned with the cutout section of the elongated body, and a distal opening; and
a second conduit having at least a portion coupled to the elongated body and extending generally parallel to the longitudinal axis thereof, the second conduit having a proximal opening and a distal opening; and
a chemical cautery stick substantially disposed within the first conduit.

13. The chemical cautery epistaxis apparatus of claim 12 further comprising,
a light conducting element substantially disposed within the second conduit.

14. The chemical cautery epistaxis apparatus of claim 12 further comprising an aperture through at least the second conduit.

15. A method of treating an epistaxis condition using the chemical cautery epistaxis apparatus of claim 13, the method comprising:
holding the chemical cautery epistaxis apparatus in a first hand;
operatively coupling the light conducting element to a light source;
inserting a proximal end of the epistaxis apparatus into a nostril;
applying light into the nostril via the light conducting element;
controlling lateral movement of the chemical cautery stick with a finger of the first hand in contact with the chemical cautery stick though the longitudinal opening of the first conduit; and
touching a cautery head of the chemical cautery stick to an inner surface of a nasal cavity.

16. A method of treating an epistaxis condition using the chemical cautery epistaxis apparatus of claim 14, the method comprising:
holding the chemical cautery epistaxis apparatus in a first hand;
operatively coupling a suction source to the distal opening of the second conduit;
inserting a proximal end of the epistaxis apparatus into a nostril;
applying suction to the nostril via the proximal end of the second conduit;
controlling a suction force of the proximal end of the second conduit with a first finger of the first hand by covering or uncovering the aperture with the first finger;
controlling lateral movement of the chemical cautery stick with a second finger of the first hand in contact with the chemical cautery stick though the cutout section of the elongated body; and
touching a cautery head of the chemical cautery stick to an inner surface of a nasal cavity.

17. A light conducting apparatus for use during treating an epistaxis condition, the light conducting apparatus comprising:
a generally frustoconical section adapted to fit substantially within a speculum;
a generally elongated cylindrical section having a circumferential flange disposed proximal the generally frustoconical section; and
an angled tip section having a light conducting tip.

18. The light conducting apparatus of claim 17, wherein the angled tip section is approximately at a 45 degree angle with respect to a longitudinal axis of the generally elongated cylindrical section.

19. The light conducting apparatus of claim 17, wherein the apparatus is comprised of a light conducting plastic.

20. A method of treating an epistaxis condition, the method comprising:
holding an epistaxis apparatus in a first hand, the epistaxis apparatus comprising,
an elongated body having (i) a proximal end, (ii) a longitudinal axis, (iii) a cutout section disposed along the length thereof, and (iv) a distal end,
a first conduit having at least a portion coupled to the elongated body and extending generally parallel to the longitudinal axis thereof, the first conduit having a proximal opening, a longitudinal opening substantially aligned with the cutout section of the elongated body for laterally moving a cautery source device therein, and a distal opening, and
a second conduit having at least a portion coupled to the elongated body and extending generally parallel to the longitudinal axis thereof, the second conduit having a proximal opening and a distal opening, the distal opening being coupled to a suction source;
inserting a proximal end of the epistaxis apparatus into a nostril;
holding an otoscope a second hand, the otoscope comprising,
a speculum, and
a light conducting apparatus secured by the speculum, the light conducting apparatus comprising,
a generally frustoconical section adapted to fit substantially within the speculum,
a generally elongated cylindrical section having a circumferential flange disposed proximal the generally frustoconical section; and
an angled tip section having a light conducting tip;
inserting at least a portion of the angled tip section into the nostril;
applying light into the nostril via the otoscope;
applying suction to the nostril via the proximal end of the second conduit;
controlling lateral movement of the cautery source device with a finger of the first hand in contact with the cautery source device though the cutout section of the elongated body; and
touching a cautery head of the cautery source device to an inner surface of a nasal cavity.
